(19) United States
(12) Patent Application Publication
(10) Pub. No.: US 2026/0011443 A1
(43) Pub. Date: Jan. 8, 2026

(54) SYSTEM AND METHOD FOR DATA GENERATION FOR USE IN DISEASE DETECTION

(71) Applicants: The University of Hong Kong, Pokfulam (HK); The Chinese University of Hong Kong, Shatin (HK)

(72) Inventors: Victor On-Kwok Li, Hong Kong (CN); Chi Kei Jacqueline Lam, Hong Kong (CN); Yang Han, Hong Kong (CN); Lawrence Yam Leung Cheung, Hong Kong (CN)

(21) Appl. No.: 19/261,145

(22) Filed: Jul. 7, 2025

Related U.S. Application Data
(60) Provisional application No. 63/668,403, filed on Jul. 8, 2024.

(51) Int. Cl. G16H 50/20

(52) U.S. Cl. CPC G16H 50/20

(57) ABSTRACT

A system generating data for use in disease detection includes a computing apparatus including a processing unit, a memory unit and a user interface. The processing unit is operatively coupled to the memory unit and the user interface. The computing apparatus is configured to receive initial disease related data, generate improved disease related data by applying a generative AI data generation framework to the initial disease related data, store the improved disease related data samples. Generating the improved disease related data includes applying each of the following data generation strategies a) observational data generation, b) cross lingual data generation and c) counterfactual data generation.

### CROSS REFERENCE TO RELATED APPLICATIONS

This application claims the benefit of and priority to U.S. Provisional Patent Application 63/668,403, filed on 8 Jul. 2024, the entire disclosure of which is incorporated herein by reference.

### TECHNICAL FIELD

The present disclosure relates to a system and method for generating data for use in disease detection, in particular, but not limited to a system and method for generating data for use detecting cognitive disease such as for example Alzheimer's disease (AD) detection or mild cognitive impairment.

### BACKGROUND

Alzheimer's disease (AD) has become a global public health concern. Low-cost and non-invasive approaches to AD detection, such as speech tests, are promising for population screening at large-scale. Previous studies have demonstrated the utility of speech tests in detecting neurodegenerative diseases such as for example in (Henderson et al. 2023; Patel et al. 2022). Recent studies have highlighted the application of artificial intelligence (AI) technologies in speech-based AD detection using audio and text data such as for example in (Li et al. 2021). Studies in 2022 showed that text embeddings from GPT-3 can distinguish AD subjects from normal controls (NCs). It has been further demonstrated, through specific studies that text embeddings combined with audio features can improve the accuracy of AD classification.

AI-driven speech-based AD detection studies have been conducted to test the effectiveness of AI-driven speech-based AD detection techniques. While these AI-driven speech-based AD studies have achieved strong performance in detecting AD dementia (near 90% accuracy on average), the accuracy of detecting its early stages, i.e., mild cognitive impairment (MCI), is still significantly lower. MCI stages are often undetected, but their detection is critical for early treatment and intervention. Detection of AD in its early stages, i.e., MCI onset, remains a challenging task, given the lack of sufficient training data and the imbalanced diagnosis labels to distinguish MCI from NC.

This data scarcity issue has generally led to biased predictions and poor generalizability in AD studies. Imbalanced AD datasets can negatively impact the predictive performance because the learned model tends to be biased towards the majority diagnosis label to minimize the overall error rate. Moreover, with limited samples and high-dimensional features, i.e., a low sample-to-feature ratio, AI-driven models are more likely to overfit the training data, leading to poor generalization to unseen data, especially from different AD cohort data. Further, AD study participants are mostly Caucasians. Other ethnicities are underrepresented. The insufficient data for different population groups may lead to biased prediction, e.g., lower sensitivity was observed for predicting MCI to AD conversion among certain minor ethnic groups.

Additionally, the sensitivity of MCI detection remains low because MCI samples are significantly fewer than normal control (NC) samples, often leading to biased predictions favoring the NC label. To date, how to reduce biases and improve generalizability in AI-driven AD studies remains an open question.

Existing AI-driven AD studies have adopted several methods to overcome the limited and imbalanced data challenges. For missing and incomplete data, simple imputation techniques (e.g., mean values) were used to preprocess the data first. Built-in imputation mechanisms were also incorporated into the model training process. Moreover, data augmentation has been adopted to tackle limited data and class imbalance problems in imaging-based AD studies, e.g., using flipped and rotated images to augment the training data for deep neural networks. Further, advanced learning techniques have been utilized to address the limited data issue. On the one hand, self-supervised learning aims to learn useful representations from data without explicit labels (such as whether AD or not), allowing AI models to learn from auxiliary tasks related to the main prediction task, such as input data reconstruction along with AD prediction. On the other hand, transfer learning, which aims to apply representations learned from one task to related tasks, can improve AD prediction limited by low-resource data. For example, capitalizing on shared text embeddings from BERT, a pre-trained language model, English corpus data has facilitated speech-based AD detection using Chinese AD corpus data. Nevertheless, most existing studies have focused on AD detection using clinical and brain imaging data instead of speech data. Utilizing limited and imbalanced speech data for AD detection in its early stages, i.e., MCI detection, remains relatively unexplored.

### SUMMARY OF THE DISCLOSURE

The present disclosure relates to a system and a computer implemented method for generating data for use in disease detection or identification. In particular, but not limited to a system and a computer implemented method for generating data for use in cognitive disease detection such as for example, Alzheimer's disease (AD) detection or mild cognitive impairment (MCI) detection. More specifically, the present disclosure relates to a system and computer implemented method for generating disease related data for use in MCI detection or identification in patients.

The present disclosure provides an improved approach to address the challenges of data scarcity and imbalance in early-stage disease detection, particularly for mild cognitive impairment (MCI). The disclosed system and method leverage a generative AI framework, utilizing one or more large language models (LLMs), to create high-quality, synthetic data that improves the performance of downstream classification models.

According to a first aspect, there is provided a computer implemented for generating data for use in disease detection comprising the steps of:
receiving initial disease related data,
generating improved disease related data by applying a generative AI data generation framework to the initial disease related data, and;
storing the improved disease related data samples.

The method for disease detection according to the first aspect, is suited for use in cognitive disease detection.

In one example the method is configured to generate the improved data for use in mild cognitive impairment (MCI) detection in patients. In another example, the method is configured to generate the improved disease related data for use in Alzheimer's disease (AD) detection in patients.

In one example, the generated improved disease related data samples may be used in a system for detecting a cognitive disease e.g., AZ detection or MCI detection. In one example, the method may be used in a framework to generate the improved disease data samples. The framework may be a machine learning network or an AI model or an AI framework.

The method is advantageous as it provides an improved output that reduces MCI prediction biases. The method or methods according to this disclosure are advantageous because they focus on improving early stage cognitive disease recognition. In particular the method or methods according to the present disclosure provide an improved process for generating improved disease related data samples that can be used in cognitive disease detection (e.g., MCI or AD).

In one example the step of generating improved data samples comprises generating improved disease related data by applying one or more large language models to the initial disease related data.

In one example step of generating the improved disease related data comprises applying one or more of the following data generation strategies:
a) observational data generation
b) cross lingual data generation
c) counterfactual data generation.

In one example the step of generating the improved disease related data comprises applying each of the following data generation strategies a) observational data generation, b) cross lingual data generation and c) counterfactual data generation.

In one example the improved disease related data comprises out-of-distribution samples comprising the same labels but with more linguistic variations from cross lingual data generation and/or out-of-distribution samples with opposite labels through counterfactual generation.

In one example each data generation strategy is implemented by a separate generative AI model executed on a computing apparatus.

In one example each data generation strategy is implemented by inputting a prompt to a generative AI model corresponding to a data generation strategy, wherein each prompt comprises a system message and a user message.

In one example each generative AI model comprises a large language model.

In one example the system message comprises a two-part message: a first part of the system message provides background information for data generation, and a second part of the system message provides detailed instructions for new data generation.

In one example wherein the user message comprises input information for data generation, wherein the input information comprises one or more of: transcription text, diagnosis labels, age, gender, race, education and MMSE information.

In one example a chain of thought (COT) prompting is used to generate the second part of the system message.

In one example observational data generation comprises the steps of:
explaining the characteristics of this text and the reasons behind why this test subject is labelled MCI,
rephrasing the original transcription to a similar but new transcription in two lines: the first line only outputs the new transcription in no more than 150 words, with a prefix ‘Text:’; the second line outputs the explanations, with a prefix ‘Explanations:’

In one example cross lingual data generation comprises the steps of:
explaining the characteristics of this text and the reasons behind why this test subject is labelled MCI,
rephrasing the original transcription to a similar but new transcription in two lines: the first line only outputs the new transcription in no more than 150 words, with a prefix ‘Text:’; the second line outputs the explanations, with a prefix ‘Explanations:’,
translating the text but not explanations into Chinese, with a prefix ‘Chinese:’.

In one example counterfactual data generation comprises the steps of:
explaining the characteristics of this text and the reasons behind why this test subject is labelled normal control (NC),
determining what characteristics a subject labelled with MCI would have, while keeping the subject's age, gender, race, and education information unchanged,
preparing a new counterfactual transcription labelled with MCI in two lines: the first line only outputs the new transcription in no more than 150 words, with a prefix ‘Text:’; the second line outputs the explanations, with a prefix ‘Explanations:’.

In one example the method comprises the step of training a text-based MCI classification model using the improved disease related data. The improved disease related data is utilized as the training data set.

In one example the step of training comprises the additional steps of:
vectorizing the improved disease related data to generate term frequency-inverse document frequency (TF-IDF) vectors by applying a frequency inverse document frequency method,
constructing text-based MCI classification by utilizing the TF-IDF vectors as inputs.

In one example, the method comprises constructing multiple text-based MCI classification models. The models may be constructed based on applying one or more of: extreme Gradient Boosting (XGBoost) or tree boosting algorithm with 100 estimators. The tree boosting algorithm may also utilize an objective function for binary logistic regression.

In one example, the method comprises the step of:
performing feature importance analysis
identifying the most important speech markers for predicting MCI with and without new data generation.

In one example the step of performing feature importance comprises calculating a feature importance score by applying Shapley Additive explanations (SHAP) analysis.

According to a further aspect, there is provided a data processing system comprising means for carrying out the method of any one of statements above. The data processing system may comprise a processing means for carrying out the method.

According to a further aspect, there is provided a computer program comprising instructions which, when the program is executed by a processing unit, cause the computing apparatus to carry out the method of any one of the statements above.

According to a further aspect there is provided a computer-readable medium comprising instructions which, when executed by a processing unit, cause the computing apparatus to carry out the method of any one of the statements above.

According to a further aspect, there is provided a system for generating data for use in disease detection comprising: a data processing unit, a memory unit operatively coupled to the data processing unit, wherein the data processing unit is configured for carrying out the method of any one the above statements.

In one example the system is configured to generate the improved data for use in mild cognitive impairment (MCI) detection in patients.

According to a further aspect, there is provided a system for generating data for use in disease detection comprising:
a computing apparatus,
the computing apparatus comprising a processing unit, a memory unit and a user interface, the processing unit is operatively coupled to the memory unit and the user interface,
the computing apparatus is configured to:
   receive initial disease related data,
   generate improved disease related data by applying a generative AI data generation framework to the initial disease related data,
   store the improved disease related data samples, and;
   wherein the step of generating the improved disease related data comprises applying each of the following data generation strategies a) observational data generation, b) cross lingual data generation and c) counterfactual data generation.

In one example the system is configured to generate the improved data for use in mild cognitive impairment (MCI) detection in patients.

In one example the computing apparatus is configured to, as part of the step of generating improved data samples, generate improved disease related data by applying one or more large language models to the initial disease related data.

In one example the computing apparatus is configured to, as part of the step of generating the improved disease related data, apply one or more of the following data generation strategies:
a) observational data generation
b) cross lingual data generation
c) counterfactual data generation.

In one example the computing apparatus is configured to apply each of the following data generation strategies a) observational data generation, b) cross lingual data generation and c) counterfactual data generation.

In one example the improved disease related data comprises out-of-distribution samples comprising the same labels but with more linguistic variations from cross lingual data generation and/or out-of-distribution samples with opposite labels through counterfactual generation.

In one example each data generation strategy is implemented by a separate generative AI model executed on a computing apparatus.

In one example each data generation strategy is implemented by inputting a prompt to a generative AI model corresponding to a data generation strategy, wherein each prompt comprises a system message and a user message. The prompt may be optionally inputted via a user interface of the computing apparatus.

In one example each generative AI model comprises a large language model. The computing apparatus may store one or more large language models in a memory unit and the processing unit is configured to apply the stored large language models to generate improved disease data.

In one example the system message comprises a two-part message: a first part of the system message provides background information for data generation, and a second part of the system message provides detailed instructions for new data generation.

In one example wherein the user message comprises input information for data generation, wherein the input information comprises one or more of: transcription text, diagnosis labels, age, gender, race, education and MMSE information.

In one example the computing apparatus is configured to apply a chain of thought (COT) prompting to generate the second part of the system message.

In one example the computing apparatus is configured to, as part of observational data generation:
explain the characteristics of this text and the reasons behind why this test subject is labelled MCI,
rephrase the original transcription to a similar but new transcription in two lines: the first line only outputs the new transcription in no more than 150 words, with a prefix ‘Text:’; the second line outputs the explanations, with a prefix ‘Explanations:’.

In one example the computing apparatus is configured to, as part of cross lingual data generation:
explain the characteristics of this text and the reasons behind why this test subject is labelled MCI,
rephrase the original transcription to a similar but new transcription in two lines: the first line only outputs the new transcription in no more than 150 words, with a prefix ‘Text:’; the second line outputs the explanations, with a prefix ‘Explanations:’,
translate the text but not explanations into Chinese, with a prefix ‘Chinese:’.

In one example the computing apparatus is configured to, as part of counterfactual data generation:
explain the characteristics of this text and the reasons behind why this test subject is labelled normal control (NC),
determine what characteristics a subject labelled with MCI would have, while keeping the subject's age, gender, race, and education information unchanged,
prepare a new counterfactual transcription labelled with MCI in two lines: the first line only outputs the new transcription in no more than 150 words, with a prefix ‘Text:’; the second line outputs the explanations, with a prefix ‘Explanations:’.

In one example the computing apparatus is configured to train a text-based MCI classification model using the improved disease related data. The improved disease related data is utilized as the training data set.

In one example, as part of the step of training, the computing apparatus is configured to:
vectorize the improved disease related data to generate TF-IDF vectors by applying a frequency inverse document frequency method,
construct text-based MCI classification by utilizing the TF-IDF vectors as inputs.

In one example, the computing apparatus is configured to construct multiple text-based MCI classification models. The models may be constructed based on applying one or more of: extreme Gradient Boosting (XGBoost) or tree boosting algorithm with 100 estimators. The tree boosting algorithm may also utilize an objective function for binary logistic regression.

In one example, the computing apparatus is configured to:
perform feature importance analysis,
identify the most important speech markers for predicting MCI with and without new data generation.

In one example the computing apparatus is configured to calculate a feature importance score by applying Shapley Additive explanations (SHAP) analysis to calculate a feature importance score.

According to a further aspect, there is provided a framework (e.g., a machine learning or AI framework) for implementing an example a method for generating data for use in cognitive disease detection comprising:
a first section is configured for or trained for data collection and pre-processing,
a second section is configured for or trained for generating data by applying one or more large language models (LLMs), wherein the data generated by the one or more LLMs comprising observational data, cross lingual data and counter factual data,
a third section is configured for or trained for cognitive disease detection and evaluation, and;
a fourth section is configured for or trained for identifying key markers for predicting a cognitive disease, wherein the cognitive disease is MCI.

In one example, the output MCI prediction as described herein may be used in AD.

In one example, the third section comprises a machine learning network or an AI model that is trained for cognitive disease detection and evaluation.

In one example, the third section is configured to or trained to:
vectorizing data samples by applying a frequency-inverse document frequency (TF-IDF) method for text vectorization,
applying a text classification model to the vectorized data samples to predict the cognitive disease. The cognitive disease may be MCI and the third section may be configured for predicting MCI by processing the vectorized data samples.

In one example the data samples may be observational data, cross lingual data and counter factual data. All three types of data i.e., all three sets of data samples may be vectorized first.

In one example, the fourth section may be trained for or configured to apply Shapely Additive Explanations (SHAP) analysis on the outputs of the third section to identify key markers for cognitive disease prediction e.g., identifying key markers for MCI prediction.

In one example, the framework may be a machine learning network or an AI model comprising at least the four sections as described herein. In one example, the framework may be a generative AI network. In one example, each LLM in the second section may be a GAI model.

In another aspect, there is provided a computer implemented method for generating data for use in mild cognitive impairment (MCI) detection comprising the steps of: receiving, by a processor, initial disease-related data comprising at least a first transcription text of a subject's speech and an associated first diagnosis label, generating, by the processor using a generative AI framework comprising one or more large language models (LLMs), improved disease related data by applying a plurality of data generation strategies to the initial disease-related data, the plurality of data generation strategies comprising: (a) observational data generation, (b) cross-lingual data generation, and; (c) counterfactual data generation, and; wherein the cross lingual data generation and the counterfactual data generation are applied to generate out-of-distribution samples, and; storing, in a memory, the improved disease-related data. The improved data may optionally be displayed on a display or a user interface.

“AD” as used in this specification is a short for Alzheimer's Disease.

“MCI” as used in this specification is short for Mild Cognitive Impairment.

“LLM” as used in the specification is short for a Large Language Model. A large language model is a machine learning model or a computational model that can comprehend and generate text. Large language models can achieve general purpose language generation and other natural language processing tasks e.g., classification. In one example an LLM can be used to comprehend and generate human language text.

“GAI” as used in this specification is short for a Generative AI. Generative AI as used in the specification is an artificial intelligence technology or model that is capable of generating text, images, videos, or other data using generative models. The generation of data may be in response to prompts or instructions.

The term “comprising” (and its grammatical variations) as used herein are used in the inclusive sense of “having” or “including” and not in the sense of “consisting only of”.

It is to be understood that, if any prior art information is referred to herein, such reference does not constitute an admission that the information forms a part of the common general knowledge in the art, in any country.

### BRIEF DESCRIPTION OF THE DRAWINGS

The accompanying drawings, which are incorporated in and constitute a part of this specification, illustrate embodiments of the disclosure and, together with the description, serve to explain the principles of the disclosure.

FIG. 1 illustrates an example of a system for generating data for use in disease detection.

FIG. 2 illustrates a schematic diagram of a computing apparatus which is arranged to be implemented as a system for generating data for use in disease detection, in particular generating data for use in MCI detection.

FIG. 3 illustrates an example method for generating data for use in disease detection.

FIG. 4 illustrates a further example method for generating data for use in disease detection.

FIG. 5 shows a framework for an example implementation of a method of generating MCI related data that is utilized in MCI detection in patients and identifying key markers for MCI prediction.

FIG. 6 illustrates a table that summarizes the descriptive statistics of the selected audio samples.

FIG. 7 illustrates an example method of the CoT prompting for Observational Generation steps.

FIG. 8 illustrates an example method of the CoT prompting Cross Lingual Generation.

FIG. 9 illustrates an example method of the CoT prompting Counterfactual Generation.

FIG. 10 illustrates table that shows the data generation statistics using different strategies and LLM models.

FIG. 11 illustrates a table that indicates the performance of MCI prediction using different data generation strategies, demonstrating their effects on facilitating model training and improving MCI prediction performance in terms of sensitivity and F1-score.

FIG. 12 illustrates a table that indicates the evaluation of different data generation combinations in improving MCI detection sensitivity and F1-score based on five-fold cross-validation.

FIG. 13 shows the top ten speech markers predicting MCI compared to NC based on the baseline model trained on the original data.

FIG. 14 shows the top ten speech markers predicting MCI compared to NC based on the best model using the original data and the counterfactual data generation.

FIG. 15 shows the top ten speech markers predicting MCI compared to NC based on the best model using the original data and all data generation.

### DETAILED DESCRIPTION OF THE PREFERRED EMBODIMENTS

Alzheimer's disease (AD) has become a global public health concern. Low-cost and non-invasive approaches to AD detection, such as speech tests, are promising for large-scale screening. Recent studies have shown that AI-driven methods can successfully detect AD dementia through audio and text data analysis. However, early detection of mild cognitive impairment (MCI) remains challenging due to insufficient training data and imbalanced diagnostic labels. The sensitivity of MCI detection remains low because MCI samples are significantly fewer than normal control (NC) samples, often leading to biased predictions favoring the NC label.

The present disclosure relates to a system and a computer implemented method for generating data for use in disease detection. In particular, but not limited to a system and a computer implemented method for generating data for use cognitive disease detection. More specifically, the present disclosure relates to a system and computer implemented method for generating disease related data for use in MCI detection in patients. The system and computer implemented method may also be used to for Alzheimer's disease (AD) detection. The computer implemented method and system described herein may be used for generating data for use in other cognitive disease detection.

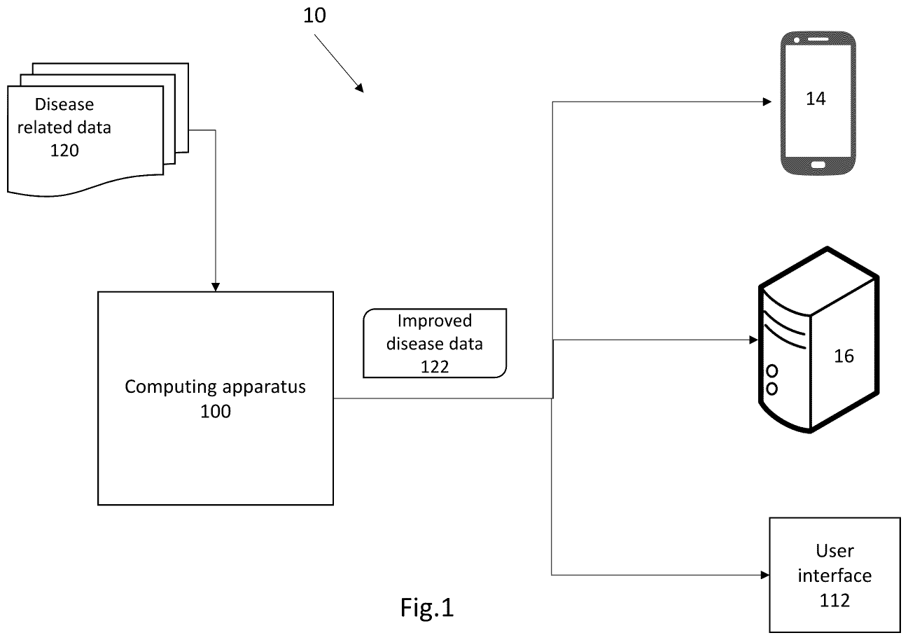

Referring to FIG. 1, an embodiment of the present disclosure is illustrated. This embodiment is arranged to provide a system 10 for generating data for use in disease detection. The system for generating data for use in disease detection comprises: a computing apparatus 100, the computing apparatus comprising a processing unit 102, a memory unit 104 and a user interface 112, the processing unit 102 is operatively coupled to the memory unit 104 and the user interface 112, the computing apparatus 100 is configured to: receive initial disease related data 120, generate improved disease related data by applying a generative AI data generation framework to the initial disease related data 122, and store the improved disease related data in a memory unit. The generated improved disease related data may be optionally displayed in the user interface 112.

In one example the step of generating the improved disease related data comprises applying each of the following data generation strategies a) observational data generation, b) cross lingual data generation and c) counterfactual data generation.

In one example the system is configured to generate the improved disease data 122 for use in mild cognitive impairment (MCI) detection in patients.

The system 10 may further optionally comprise other components such as for example, a clinician device 14 or a disease identification server 16. The outputs from the computing apparatus 100 (i.e., the improved disease data 122) may be transmitted to one or more components such as the user interface 112, clinician device 14 or server 16.

The clinician device 14 may be a mobile device (e.g., smartphone or tablet) associated with a patient's physician e.g., a primary care physician or a neurologist or other clinician. The clinician device 14 may be adapted for MCI detection of patients using the improved disease data 122 generated by the computing apparatus 100.

The disease identification server 16 may be a hospital server or an electronic medical records (EMR) server. The disease identification server 16 may be a government health server storing patient data or another server that is adapted to store patient clinical information. The server 16 may be further configured to utilize the improved disease data 122 and determine a patient suffering a disease. In one example, the server 16 may be configured to use the improved disease data 122 for MCI detection in patients.

The clinician device 14 or server 16 may be adapted to implement one or more models for MCI detection. The MCI detection output may be stored in the server 16 or may be displayed to a clinician.

The computing apparatus 100 is configured to generate improved disease related data 122. The improved disease related data 122 can be utilized to train one or more mild cognitive impairment (MCI) detection models. The MCI detection models can be used to detect a patient suffering from MCI by processing new input data. The improved disease related data may be indicative of MCI in patients.

In one example the generated improved disease related data 122 may be transmitted from the computing apparatus 100 to other devices in the system 10 via a wireless network e.g., cellular network or Wi-Fi or by a wired connection. The devices such as clinician device 14 and medical server 16 and the computing apparatus 100 may be part of a network such as a hospital network. The MCI detection models may be executed on other devices such as the clinician device 14 or the medical server 16 or on another server.

In one example embodiment, the computing apparatus 100 comprises a processing unit, a memory unit and an appropriate user interface. The computing apparatus (i.e., computer or computing device or processing device or computer system) may be implemented by any computing architecture, including portable computers, tablet computers, stand-alone Personal Computers (PCs), smart devices, Internet of Things (IoT) devices, edge computing devices, client/server architecture, “dumb” terminal/mainframe architecture, cloud-computing based architecture, or any other appropriate architecture. The computing device may be appropriately programmed to implement the disclosure.

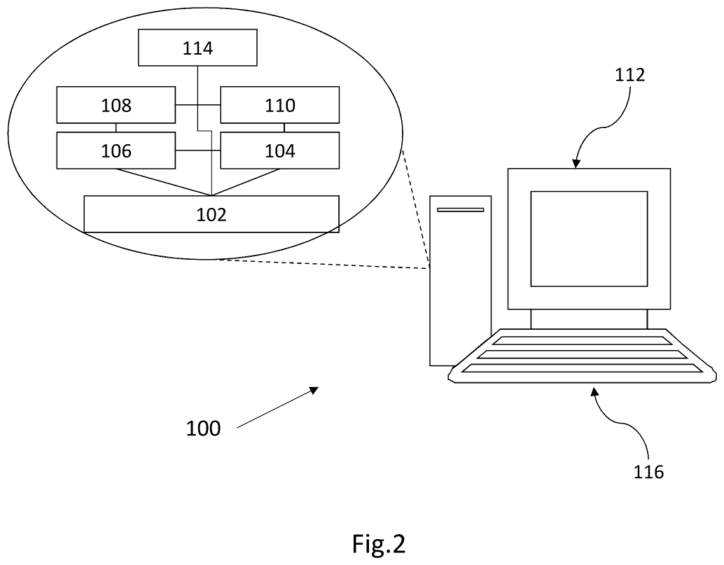

As shown in FIG. 2 there is a shown a schematic diagram of a computing apparatus or computer server 100 which is arranged to be implemented as an example embodiment of a system for generating data for use in disease detection or disease state detection. The system is adapted for generating data for use in MCI detection in a patient.

In this embodiment the system comprises a computing apparatus 100 (or compute server) which includes suitable components necessary to receive, store and execute appropriate computer instructions. The components may include a processing unit 102, including Central Processing Unit (CPU), Math Co-Processing Unit (Math Processor), Graphic Processing Unit (GPUs) or Tensor processing unit (TPUs) for tensor or multi-dimensional array calculations or manipulation operations, one or more memory units such as for example a read-only memory (ROM) 104 and a random-access memory (RAM) 106. The computing apparatus 100 comprises input/output devices such as disk drives 108, input devices 110 such as an Ethernet port, a USB port, etc.

The computing apparatus comprises a user interface 112. The user interface may comprise a display 112 such as a liquid crystal display, a light emitting display or any other suitable display and optionally a keypad 116 or other elements to allow a user to input instructions. The display 112 may be a touchscreen display to allow a user to input commands or data and the display may further be configured to present outputs to a user. The user interface 112 may be optional. The computing apparatus 100 comprises one or more communications links 114.

The computing apparatus 100 may include instructions that may be included in ROM 104, RAM 106 or disk drives 108 and may be executed by the processing unit 102. The stored instructions may allow the computing apparatus 100 to perform one or more functions. The computing apparatus 100 may be configured to execute the stored instructions to perform the steps of identifying AD causing genes. The instructions may be coded using an appropriate coding language such as for example C++ or C.

There may be provided a plurality of communication links 114 which may variously connect to one or more computing devices such as a server, personal computers, terminals, wireless or handheld computing devices, Internet of Things (IoT) devices, smart devices, edge computing devices. At least one of a plurality of communications link may be connected to an external computing network through a telephone line or other type of communications link. The communications link 114 may be Wi-Fi module, Bluetooth module or a cellular module.

The computing apparatus 100 may include storage devices such as a disk drive 108 which may encompass solid state drives, hard disk drives, optical drives, magnetic tape drives or remote or cloud-based storage devices. The computing apparatus 100 may use a single disk drive or multiple disk drives, or a remote storage service. The server 100 may also have a suitable operating system which resides on the disk drive or in the ROM of the apparatus 100.

The computer or computing apparatus 100 may also provide the necessary computational capabilities to operate or to interface with a machine learning network, such as a neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time. The computing apparatus 100 comprises computational capabilities to execute generative AI models and large language models (LLMs).

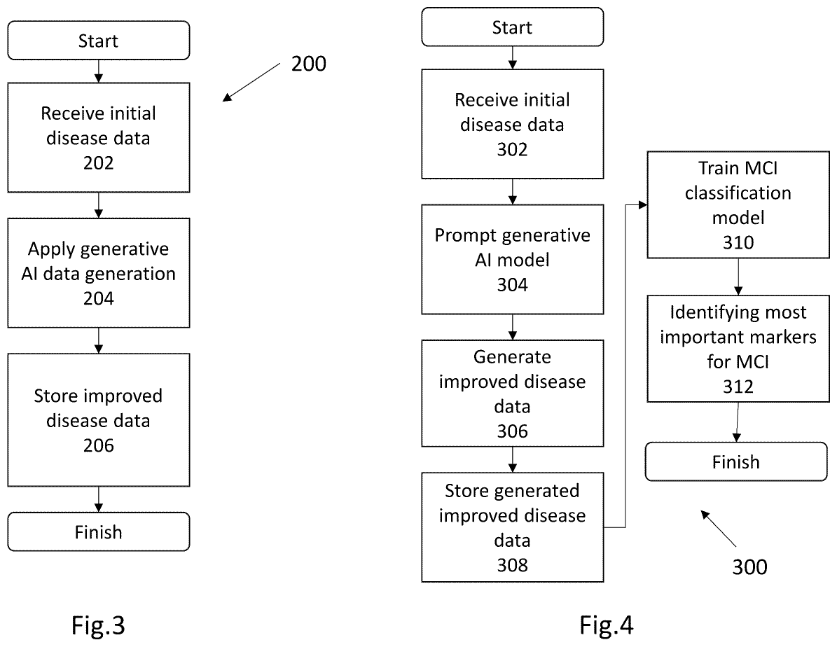

FIG. 3 illustrates an example embodiment of a computer implemented method for generating data for use in disease detection comprising the steps of: receiving initial disease related data 202, generating improved disease related data by applying a generative AI data generation framework to the initial disease related data 204, and; storing the improved disease related data samples 206.

FIG. 4 illustrates a further example embodiment of a computer implemented method 300 for generating data for use in disease detection, in particular MCI detection. The method 300 is an example method for generating data for use in MCI detection.

Referring to FIG. 4, the method 300 commences at step 302. Step 302 comprises receiving initial disease related data 202. The disease data may be data related to AD, in particular MCI related data. The initial data may be received from a database e.g., DementiaBank database. The data may be speech-based data and labelled as NC (normal control) and MCI (mild cognitive impairment).

Step 304 comprises inputting one or more prompts to a generative AI model. The method may comprise prompting multiple generative AI models, wherein each generative AI model corresponds to a particular data generation strategy. The generative AI models provide a generative AI based data generation framework. Each model may be adapted to generate data utilizing a specific data generation strategy. Each data generation strategy is implemented by a separate generative AI model executed on a computing apparatus.

In one example, each prompt comprises a system message and a user message.

In one example, each generative AI model may comprise a Large Language Model (LLM) adapted to generate data. Each data generation strategy may be executed by a unique LLM.

The system message comprises a two-part message: a first part of the system message provides background information for data generation, and a second part of the system message provides detailed instructions for new data generation.

Step 306 comprises generating improved disease related data using the one or more data generation strategies. In one example, at least two data strategies are applied to generate the data at step 306. Three LLMs may be adapted to generate data, wherein each LLM may be adapted to generate data by applying one of the two data generation strategies.

At step 306 the at least two data generation strategies applied to generate at least two sets of data comprise cross lingual data generation and counterfactual data generation. The improved disease related data (i.e., improved disease related data set) comprises both cross lingual data and counterfactual data.

Step 308 comprises storing the generated improved disease related data. Optionally, the method may comprise transmitting the improved disease related data to one or more other devices.

Step 310 comprises training a text-based MCI classification model using the improved disease related data. The improved disease related data is utilized as the training data set.

In one example the step of training may comprise the additional steps of: vectorizing the improved disease related data to generate TF-IDF vectors by applying a frequency inverse document frequency method and constructing text-based MCI classification by utilizing the TF-IDF vectors as inputs.

In one example, the method 300 may comprise constructing multiple text-based MCI classification models. The models may be constructed based on applying one or more of: extreme Gradient Boosting (XGBoost) or tree boosting algorithm with 100 estimators. The tree boosting algorithm may also utilize an objective function for binary logistic regression.

Step 312 comprises identifying the most important speech markers for predicting MCI. Step 312 may comprise identifying the most important speech markers for predicting MCI with and without new data generation.

The method steps 310, 312, may be optional in the method. The method 300 may comprise steps 302 to 308. The method 300 may be repeated one or more times. The method 300 is preferably executed by the computing apparatus 100 as described herein.

The methods 200 and 300 may be embodied as executable instructions and may be stored in a memory unit 104, 106. The memory may be a non transitory computer readable medium. The processor 102 may be programmed to execute the method 200 or 300 by executing the stored instructions embodying the method 200 or 300.

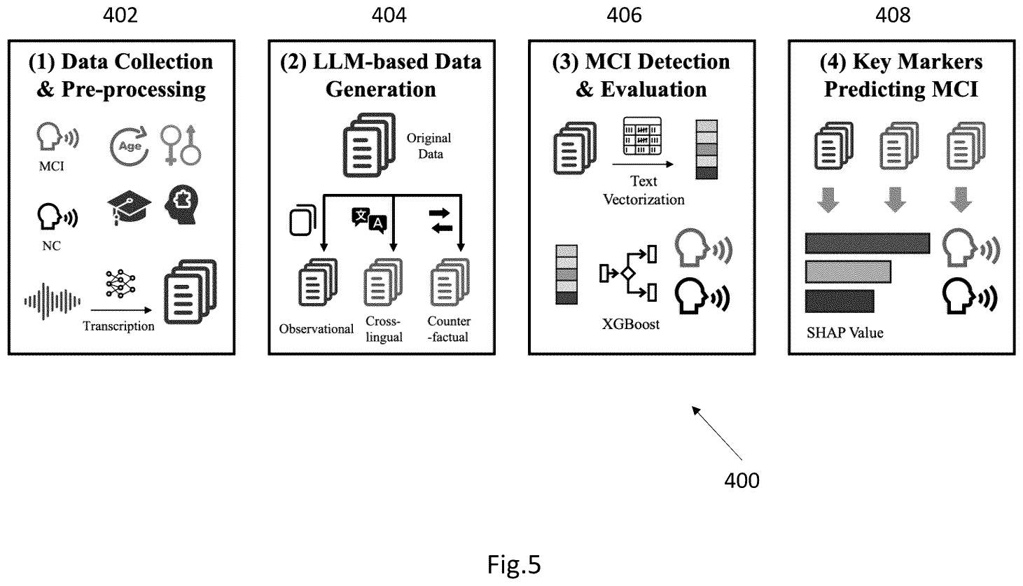

FIG. 5 shows a framework 400 for an example implementation of a method of generating MCI related data that is utilized in MCI detection in patients and identifying key markers for MCI prediction. The framework 400 comprises a first section 402 that comprises data collection and pre-processing. Section 404 comprises generating data by utilizing one or more LLM based data generation strategies. Section 406 comprises MCI detection and evaluation by a model. Section 408 comprises identifying key markers for predicting MCI.

Below is described an example implementation of a method for generating disease related data, in particular a method for generating MCI related data. The example implementation also included testing the improved disease related data generated from the method for data generation for MCI detection. The test results are also discussed below.

The foregoing example details the proposed method in four steps: (1) speech data collection and pre-processing, (2) LLM-based text data generation using different strategies: (a) observational generation, (b) cross-lingual generation, and (c) counterfactual generation, (3) text-based MCI classification model training and evaluation, and (4) feature importance analysis to identify the most important speech markers predicting MCI without and with new data generation incorporated.

### Data Collection and Pre-Processing

For the example implementation as a first step data is collected and pre-processed. In one example implementation Pitt dataset was downloaded from the DementiaBank database. The Pitt dataset is based on a longitudinal AD study where subjects were followed up in multiple years. Given the focus of this implementation on speech-based MCI detection, all subjects with speech data available and labelled with NC and MCI were included. According to the description of the Pitt dataset, NC is defined by the diagnosis category coded 8 (800 or 821), and MCI is defined by the diagnosis category coded 6 or 7 (600, 610, 611, 720, or 740).

Baseline demographics, including age, gender, race, and education (in years), were collected. Baseline and follow-up cognitive scores derived from the Mini-Mental State Examination (MMSE) were also collected. Further, and most importantly, the baseline and follow-up audio recordings of the Cookie Theft test, the most used picture description test in clinical settings, were collected. Subjects with NC and MCI labels were selected, including 89 NC and 18 MCI subjects. Since the longitudinal observations were sparse and limited, the data points were considered cross-sectional samples to enlarge the sample size, resulting in 129 audio samples labelled with NC and 24 with MCI. Among these 153 samples, all demographic information was complete, and the missing ratio of MMSE was 0.7%.

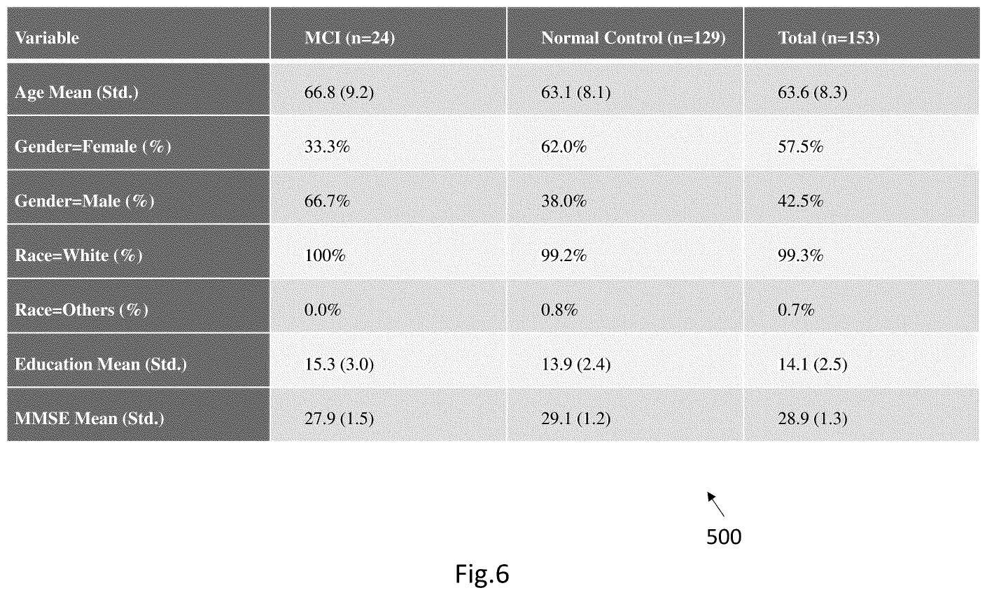

The table 500 in FIG. 6 summarizes the descriptive statistics of the selected audio samples. As shown in the table 500 of FIG. 6, the difference in MMSE was small, suggesting that MMSE may not be a good indicator to distinguish MCI from NC and detect the subtle changes in cognitive function in the early stages of AD.

The collected audio recordings in English were transcribed to text data using OpenAI's Whisper model (Radford et al. 2023), a text-to-speech model pre-trained on a large amount of audio data. In addition to the audio input, a simple text prompt, “Umm, let me think like, hmm . . . . Okay, here's what I'm, like, thinking.”, was provided to the model to capture the filler words in the transcript according to the official documentation for speech-to-text prompting (OpenAI n.d.-c). Non-English characters in the resulting transcriptions were removed before further analysis. Finally, a tabular dataset with seven columns, including age, gender, race, education, MMSE, transcription text, and diagnosis label, was generated.

### LLM Based Data Generation Framework

After data collection and pre-processing, an LLM-based data generation framework may be developed and applied to enlarge the pre-processed text dataset, which was of small sample size and highly imbalanced (24 MCI samples out of 153 samples), by generating more text samples that are synthetic yet realistic, leveraging on the prior knowledge encoded in LLMs pre-trained on the massive amount of data. The newly generated samples were used for model development only, not model evaluation.

Specifically, the LLM based data generation framework comprises applying three data generation strategies, including (1) observation data generation, (2) cross-lingual data generation, and (3) counterfactual data generation. Observation data generation aims to generate new MCI-like text samples based on the observed MCI samples from the existing dataset. Observational data generation is adapted to generate MCI-like text samples e.g., by using an LLM. Building on top of the observational data generation strategy, two novel generation strategies were incorporated into the framework. On the one hand, cross-lingual data generation further translates the new MCI-like samples into another language (Chinese in this example), introducing cross-linguistic diversity in the training data and facilitating out-of-distribution learning. On the other hand, counterfactual data generation aims to generate new MCI-like text samples based on the observed NC samples from the existing dataset while controlling for other variables as much as possible. The counterfactual generation process requires a deeper understanding of the disease mechanism, exploiting the causal knowledge encoded in LLMs, to answer a what-if question, such as what the speech observed from the NC subject would be if he/she was an MCI subject, while holding other information unchanged.

Each LLM-based data generation strategy was implemented through prompting engineering using OpenAI's text generation application programming interfaces (APIs) (OpenAI n.d.-b). The prompt consists of two parts: a system message and a user message. The system message is optional and contains instructions on how the AI-driven conversation system should behave, e.g., how to rephrase the text. The user message gives a specific request for the AI system to respond, e.g., a text example (OpenAI n.d.-b). In this example implementation, the input to the OpenAI API was formatted with a two-part system message first, followed by a user message. Specifically, the first part of the system message provides background information for the data generation task.

Use the following step-by-step instructions to respond to user inputs. The user inputs are related to the transcription of one test subject describing the Cookie Theft picture from the Boston Diagnostic Aphasia Exam. Other information of the test subject is provided, including, age, gender, race, education level (number of years), and Mini Mental State Examination (MMSE) score. Before the step-by-step instructions, some background information is listed as follows. This Cookie Theft picture description task is used to determine whether one is probable Alzheimer's disease (AD), mild cognitive impairment (MCI), or normal control (NC). The MMSE score measures one's cognitive function but needs adjustment for the education level. The step-by-step instructions are listed as follows.

The second part of the system message provides detailed step-by-step instructions for new data generation. The step-by-step instructions were designed based on the chain-of-thought (CoT) prompting strategy, i.e., a sequence of intermediate reasoning steps between the input and output. This prompting method has enabled significant improvement in the reasoning ability of LLMs on some tasks, such as math problems.

Three data generation strategies were designed and implemented. The first prompting strategy, observational generation, produces new samples based on the observed dataset, mimicking the existing samples to generate similar samples. Building on top of the first one, the second prompting strategy, cross-lingual generation, takes a further step by translating the mimicked samples into another language, aiming to introduce more linguistic variations in the monolingual dataset and allow for out-of-distribution learning. The third prompting strategy, counterfactual generation, deviates from the other methods and exploits the causal reasoning ability of LLMs to generate examples opposite to the observed samples.

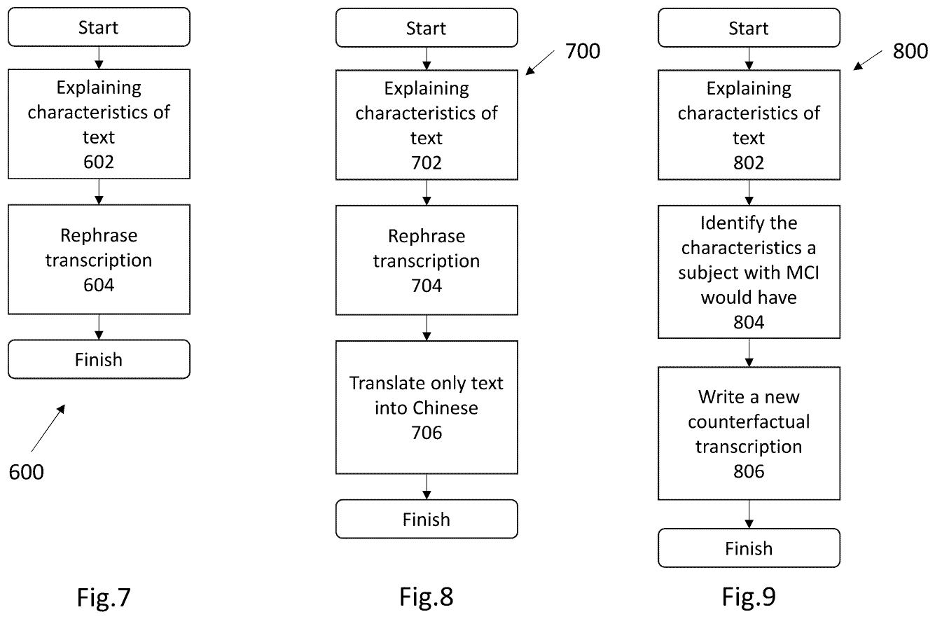

FIG. 7 illustrates an example method of the CoT prompting for Observational Generation 600. Method 600 is an example method of observational data generation. Step 602 comprises explaining the characteristics of this text and the reasons behind why this test subject is labelled MCI. Step 604 comprises given the explanations from Step 602, rephrase the original transcription to a similar but new transcription in two lines: the first line only outputs the new transcription in no more than 150 words, with a prefix ‘Text:’; the second line outputs the explanations, with a prefix ‘Explanations:’

In one example, observational data generation comprises generating a second transcription (i.e., new transcription) text that mimics linguistic characteristics of the original transcription (i.e., first transcription). Observational data generation further comprises associating the second transcription text with the first diagnosis label.

FIG. 8 illustrates an example method of the CoT prompting Cross Lingual Generation 700. Method 700 is an example method of Cross Lingual Data generation. Step 702 comprises explaining the characteristics of this text and the reasons behind why this test subject is labelled MCI. Step 704 comprises, given the explanations from Step 702, rephrase the original transcription to a similar but new transcription in two lines: the first line only outputs the new transcription in no more than 150 words, with a prefix ‘Text:’; the second line outputs the explanations, with a prefix ‘Explanations:’. Step 706 comprises, given Step 704, only translating the text but not explanations into Chinese, with a prefix ‘Chinese:’

In one example, cross lingual data generation comprises translating the second transcription into a target language e.g., Chinese or any other language. The target language may be different from the source language of the original transcription (i.e., first transcription) to create a translated transcription.

FIG. 9 illustrates an example method of the CoT prompting Counterfactual Generation 800. Method 800 is an example method of counterfactual data generation. Step 802 comprises explaining the characteristics of this text and the reasons behind why this test subject is labelled NC. Step 804 comprises, given the explanations from Step 802, imagine or identify the characteristics a subject labelled with MCI would have, while keeping the subject's age, gender, race, and education information unchanged. Step 806 comprises, given the reasons from Step 804, write a new counterfactual transcription labelled with MCI in two lines: the first line only outputs the new transcription in no more than 150 words, with a prefix ‘Text:’; the second line outputs the explanations, with a prefix ‘Explanations:’.

In one example, counterfactual generation comprises generating a third, counterfactual transcription based on the original transcription (i.e., first transcription) associated with a first diagnosis label. The counterfactual transcription may exhibit linguistic characteristics of a second diagnosis label that is opposite to the first diagnosis label.

Next, the user message may be provided as the input for data generation. The input described an example, including transcription text, diagnosis label, age, gender, race, education, and MMSE information. Given LLM's capability in handling missing tabular inputs (Borisov et al. 2022), missing values were not imputed during data pre-processing but represented by the string “MISSING”.

The original transcription of the test subject is given as follows: <transcription text>.” The label of this transcription is: <diagnosis label>. The test subject's age is <age>, gender is <gender>, race is <race>, education level (number of years) is <education>, and MMSE score is <MMSE score>.

GPT-3.5-Turbo and GPT-4 models were tested for data generation using the default parameters listed in OpenAI's API documentation (OpenAI n.d.-a). Two open-source LLMs, including Gemma-2 (with 9 billion parameters) (Mesnard et al. 2024) and Llama-3.1 (with 8 billion parameters) (Touvron et al. 2023), were tested for data generation using the recommended configurations that can improve both the quality and diversity of generated text (temperature=1.5; min-p=0.1) (Nguyen et al. 2024). The data generation targeted a new set of MCI samples with a similar size to the NC samples. For observational and cross-lingual data generation based on existing MCI samples, the data generation process (i.e., one iteration of all samples targeting for generation) was repeated five times to match the number of NC samples. In contrast, the data generation process only needed to be performed once for counterfactual generation based on existing NC samples.

After each type of LLM-based data generation, unsupervised clustering-based outlier detection was performed, given that low-quality data samples could have negative impacts on downstream prediction tasks (Seedat et al. 2023). The local outlier factor algorithm (Cheng et al. 2019), which computes outlier scores based on the deviation of a data point with respect to its nearest K neighbors, was adopted using the default parameters (K=20; threshold=0.1). The identified outliers were removed before MCI predictive modeling.

The methods 600, 700 and 800 may be performed or executed by the computing apparatus 100. The methods 600, 700 and 800 may be stored as executable instructions in a memory unit (104, 106) of the computing apparatus 100 and the methods 600, 700, 800 may be executed by the processor 102.

### Text Classification Model Training and Evaluation

The original and generated text samples were first vectorized before model training and evaluation. The term frequency-inverse document frequency (TF-IDF) method was adopted for text vectorization. Unlike advanced text embedding methods, which generate a high-dimensional vector for each text sample based on deep learning models such as transformers, the traditional TF-IDF method was selected to obtain more explainable features to be investigated in feature importance analysis, allowing for a deeper understanding of how data generation can help alleviate biases in MCI prediction.

Irrelevant keywords were removed, and filler words were unified before TF-IDF vectorization. Specifically, irrelevant keywords included “okay”, “alright”, “tell”, “see”, “describe”, “picture”, “action”, and “happening”. These words were often observed at the beginning of the recording, asking the test subject to perform the picture description task, e.g., “I want you to tell me all of the action that you see, okay?”. Punctuations and filler words, including “ . . . ”, “uh”, “um” (“umm” and “ummm”), and “hm” (“hmm” and “hmmm”), were converted to a unified representation (a single keyword “PAUSE”) to capture the pauses and hesitations in speech data. Moreover, Chinese text (from cross-lingual generation) was tokenized using a Chinese text segmentation software (Sun n.d.).

Taking the TF-IDF vectors as the inputs, text classification models were constructed based on extreme Gradient Boosting (XGBoost), a tree boosting algorithm with 100 estimators using an objective function for binary logistic regression. MCI classification models were trained across various scenarios: (1) the original data, (2) the original data plus the three disclosed data generation strategies, and (3) the original data plus different combinations of data generation strategies. For the XGBoost model trained on the original data, the parameter to control the balance of positive and negative weights for imbalanced labels was set to the ratio of original NC and MCI samples. This parameter was set to the default value of one for other scenarios with newly generated MCI samples to overcome the limited MCI samples in the dataset.

Five-fold cross-validation was performed to evaluate the performance of MCI classification models using text data. Each fold was a 20% subsample of the original dataset. Four folds were selected as the training set and the remaining one as the testing set. LLM-based data generation further enlarged the original training set while the testing set remained held out for model evaluation. In other words, the newly generated samples were used for model training only. This cross-validation procedure was repeated five times to ensure that all folds were used for testing. As a result, each MCI sample was tested precisely once, thus providing a better understanding of MCI classification performance, even though the number of MCI samples was limited. The same cross-validation folds were applied to each MCI classification model under different data generation strategies.

Two performance evaluation metrics, including sensitivity and F1-score, were adopted in this example implementation. Sensitivity (also known as recall or true positive rate) measures the true positive rate. F1-score provides a more balanced view of predictive accuracy given imbalanced labels. However, the traditional accuracy metric, which measures the percentage of correctly labelled samples, was not used because it was biased towards imbalanced labels. For example, given the imbalanced distribution of positive and negative samples in this implementation (24 MCI and 129 NC samples), a naive classifier that always predicts NC can still give an 84% accuracy but fails to predict MCI every time, making it not useful for AD screening at all.

The two metrics used in this example implementation are further elaborated in Equations (1) and (2). Average sensitivity and F1-score across the five-fold cross-validation were reported in this example implementation. The higher the sensitivity and F1-score values, the better the predictive performance.

$$Sensitivity = TP / ( TP + FN ) \qquad ( 1 )$$

$$F 1 - score = \frac{TP}{TP + \frac{1}{2} ( FP + FN )} \qquad ( 2 )$$

where TP, FN, and FP are obtained from the confusion matrix calculated based on the predicted and ground truth labels, representing the number of true positives (MCI labels that are correctly predicted), false negatives (MCI labels that are incorrectly labelled), and false positives (NC labels that are incorrectly labelled), respectively.

### SHAP Analysis for Identifying the Most Important Speech Markers.

In one example the step of performing feature importance comprises calculating a feature importance score by applying Shapley Additive explanations (SHAP) analysis.

After model evaluation, feature importance analysis was performed to investigate the key speech features predicting MCI compared to NC, allowing for a better understanding of the new insights brought by LLM-based text data generation and why the biases in MCI prediction can or cannot be reduced via new data generation. The feature importance score was calculated using SHapley Additive explanations (SHAP) analysis.

In general, SHAP analysis uses a cooperative game theoretic approach to allocate credits for a model's output among its input features. Here, game theory is connected to AI by matching input features with players in a game and aligning the model function with the game's rules. A feature participates in a model when information is available about its value. The importance of each feature is quantified by its contribution to the model prediction across all possible orderings of feature coalitions.

Three models were selected for SHAP analysis, including (1) the baseline model trained on the original data, (2) the best model trained based on one of the three data generation strategies, and (3) the best model trained based on the best combination of data generation strategies. For XGBoost models, tree-based SHAP analysis was performed. The SHAP value of each feature was calculated, utilizing both training and testing sets. Features were ordered by their SHAP values, highlighting their high impacts on individual samples using the maximum absolute SHAP value, i.e., features having significant positive or negative effects on MCI onset.

The example implementation was assessed and evaluated. Evaluation results of different data generation strategies and the most important speech markers distinguishing MCI compared to NC are presented below.

### Evaluation of Data Generation Strategies

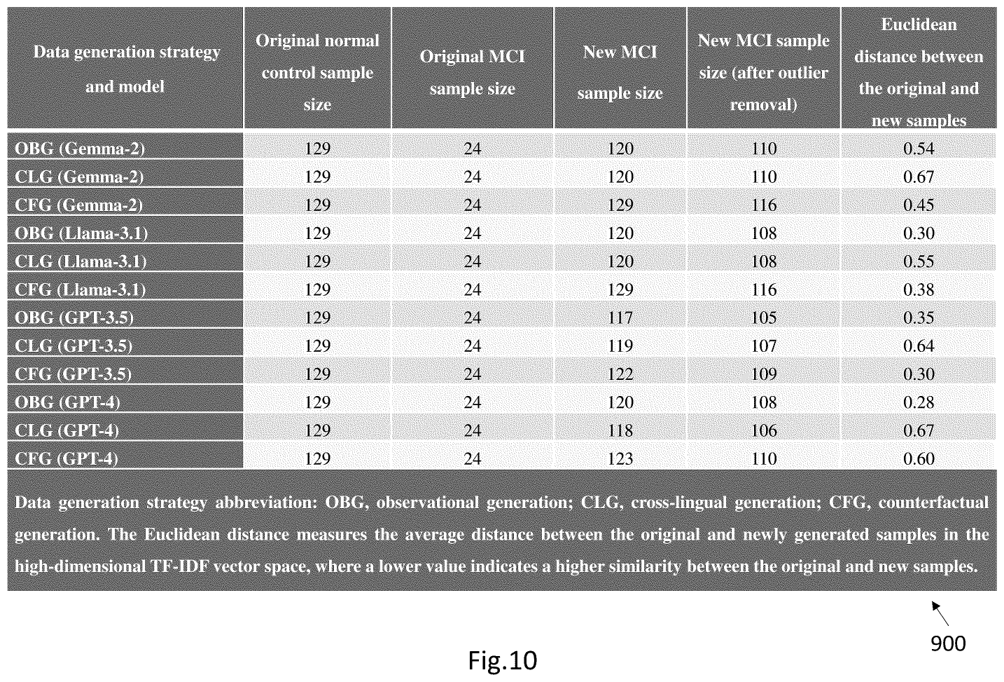

FIG. 10 illustrates table 900. Table 900 shows the data generation statistics using different strategies and LLM models. The expected number of new MCI samples obtained from observational and cross-lingual generation is 120, given that these two strategies were repeated five times. The expected number of new MCI samples obtained from counterfactual generation is 129 (i.e., the number of NC samples). However, a few samples were dropped, mainly due to the following reasons: (1) some generated text samples failed to use the specific format provided in the prompt, and no new transcription data could be extracted, and (2) some were automatically blocked due to the potentially sensitive content according to the content filtering of Microsoft Azure OpenAI service.

In one example, for each data generation strategy across different LLM models (including Gemma-2, Llama-3.1, GPT-3.5, and GPT-4), experiments were performed by calculating two mean text vectors based on the original and newly generated samples using the TF-IDF method. The average distance was calculated using the Euclidean distance between the two mean text vectors. Results show that the average distance varied across different data generation strategies. Observational data generation, which aims to mimic patterns observed in the original samples without introducing substantial variations, has yielded the smallest distances on average, especially with GPT-4, indicating that the generated samples are more consistent with the original samples. One exception is Gemma-2, which shows a relatively higher distance compared to other models, suggesting that it may struggle to replicate the original data distribution closely. Cross-lingual data generation has yielded the largest distances likely due to the variations introduced by cross-lingual transformations. This finding is consistent across different models, suggesting that such variations are more likely to be driven by the data generation strategy rather than the model's generation capabilities. In terms of counterfactual data generation, GPT-4 and Llama-3.1 have generated samples with higher distances from the original ones, suggesting that these models are more capable of generating complex and diverse samples in hypothetical scenarios.

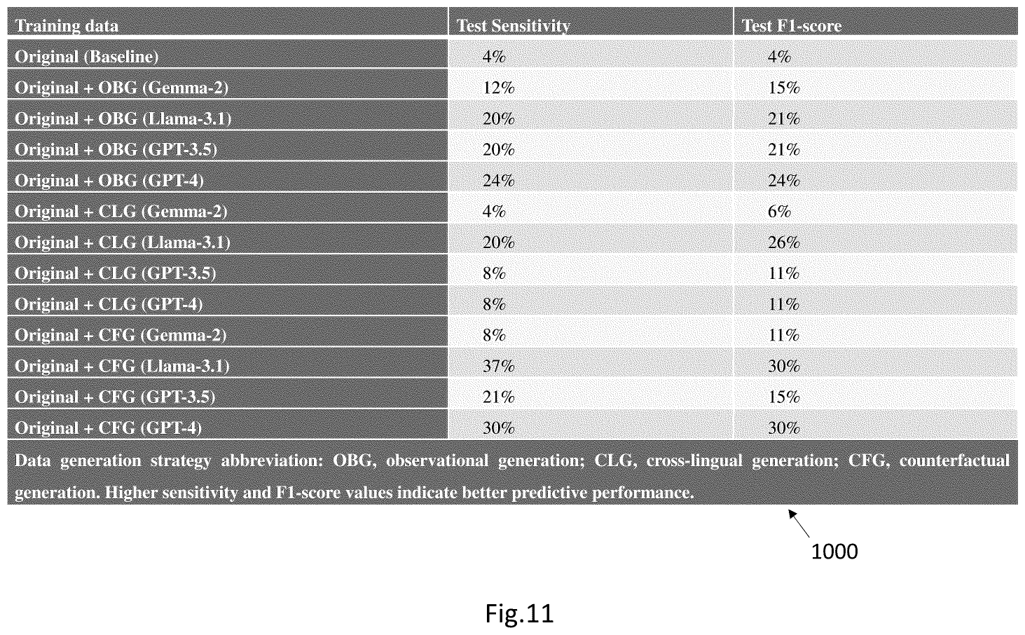

Five-fold cross-validation was performed based on the original and newly generated data. The generated data were used only for model training. FIG. 11 illustrates table 1000. Table 1000 indicates the performance of MCI prediction using different data generation strategies, demonstrating their effects on facilitating model training and improving MCI prediction performance in terms of sensitivity and F1-score. All data generation methods have achieved higher predictive performance than the baseline model using the original data only. GPT-4 consistently delivered robust performance across all strategies, particularly excelling in observational generation while maintaining a high F1-score in counterfactual generation. Among open-source LLMs, Llama-3.1 was shown in some example tests to demonstrate exceptional performance in counterfactual generation, while Gemma-2 performed less well than other models across all data generation strategies. Specifically, among LLMs employing observational generation, GPT-4 achieved the best sensitivity (24%) and F1-score (24%), outperforming GPT-3.5 and open-source models, including Gemma-2 and Llama-3.1. In terms of cross-lingual generation, performance varied across LLMs.

Llama-3.1 outperformed all other models, achieving a sensitivity of 20% and the highest F1-score of 26%. Gemma-2 showed minimal improvement, while GPT models were tied at modest values of 8% sensitivity and 11% F1-score. The counterfactual generation strategy demonstrated the greatest potential for improving sensitivity. Llama-3.1 achieved the highest sensitivity (37%) and an F1-score of 30%, matching GPT-4's F1-score, although GPT-4 trailed slightly in sensitivity (30%). GPT-3.5 and Gemma-2 performed less competitively, with GPT-3.5 reaching a sensitivity of 21% and an F1-score of 15%.

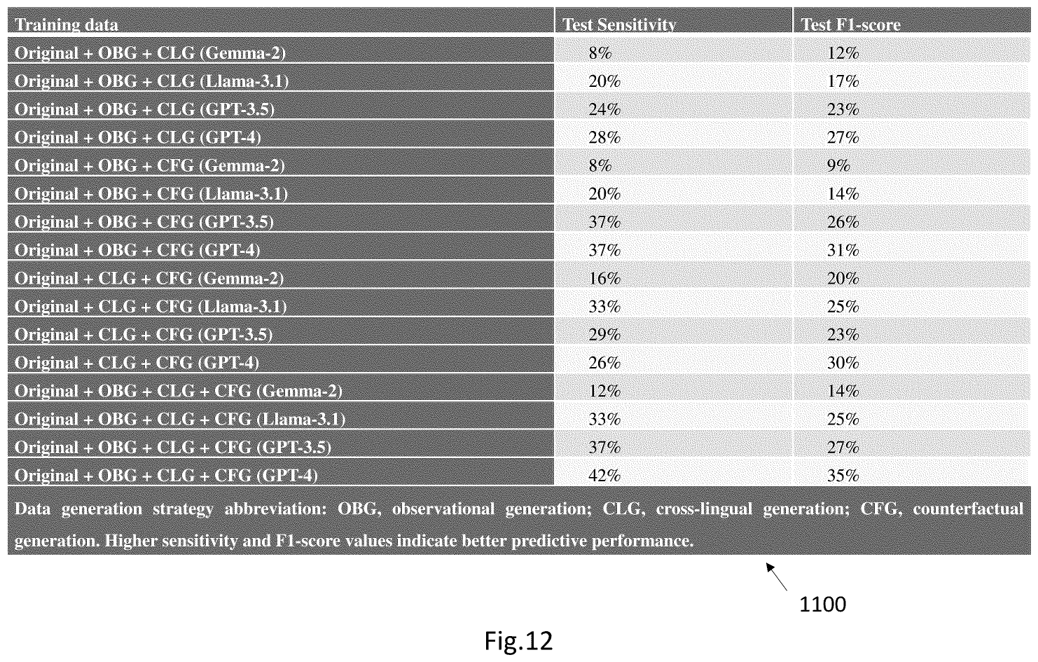

Moreover, the counterfactual generation has outperformed other data generation methods, especially when using Llama-3.1 and GPT-4 models (which have generated counterfactual samples with higher distances from the original ones, as shown in Table 900). This advantage may be attributed to its reliance on causal reasoning, which enables the introduction of hypothetical “what-if” scenarios and reduces spurious correlations between observed features and their labels (Ilse et al. 2021). This process has been shown to effectively address dataset biases, enhancing out-of-distribution generalization performance in computer vision tasks such as imaging classification (Chang et al. 2021) and language processing tasks such as question-answering (Sachdeva et al. 2023). In contrast, the cross-lingual generation has only achieved the lowest improvement on the baseline. One possible reason is that the simple TF-IDF method may only partially capture the cross-lingual text representations. Although the TF-IDF vector is a more interpretable representation, it may fail to capture the contexts and semantic relationships compared to more advanced methods, such as deep learning-based multilingual text representation learning (Cahyawijaya et al. 2024). Nonetheless, as shown in Table 900, it can still provide new samples that are different from the original monolingual samples. FIG. 12 illustrates table 1100. Table 1100 illustrates the evaluation of different data generation combinations in improving MCI detection sensitivity and F1-score based on five-fold cross-validation.

As shown in these examples, these example combinations generally led to improvements over single-strategy approaches, with notable variations among models. The best performance has been achieved when using all three data generation methods together based on GPT-4 (see Table 1100). In contrast, for the Llama model, combining multiple strategies did not surpass the performance achieved with counterfactual generation alone. Meanwhile, the Gemma model achieved its best performance when combining cross-lingual and counterfactual generation. These findings highlight the additive effect of data generation strategies on improving predictive performance, particularly with GPT models. Compared to Gemma and Llama models, GPT models are better equipped to capture diverse aspects of speech-based MCI detection, introducing beneficial variations into the training data that enhance model generalization and robustness.

Key Speech Markers Distinguishing MCI from NC

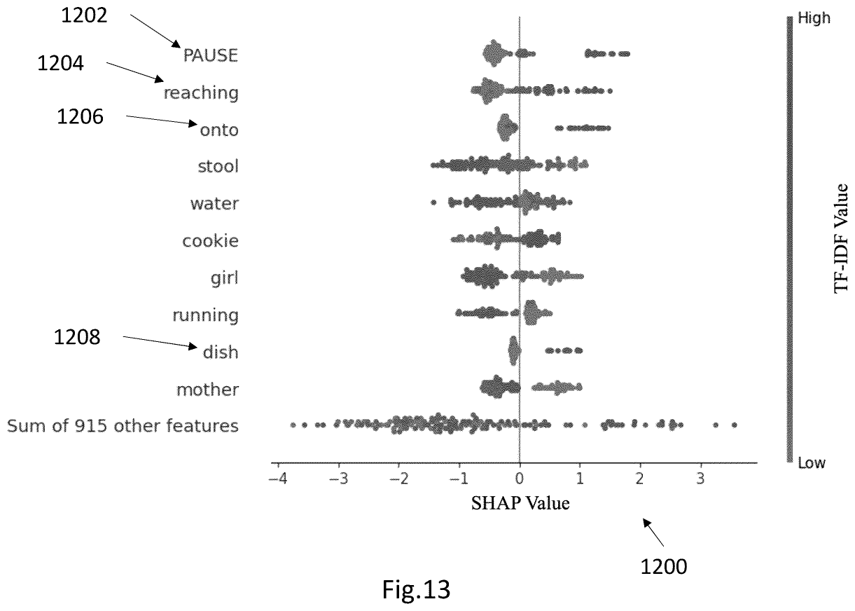

In addition to performance evaluation, SHAP analysis was performed to understand which speech markers are most important in MCI prediction before and after data generation. This feature importance analysis can facilitate understanding why LLM-based data generation can improve predictive performance and reduce biases, bringing new insights into the key speech markers predicting MCI. FIG. 13 shows the top ten speech markers predicting MCI compared to NC based on the baseline model trained on the original data.

Referring to FIG. 13, the top ten speech markers predicting MCI compared to NC based on the baseline model trained on the original data are illustrated in plot 1200. Each circle in the plot represents one sample. The color or shade of the circle indicates the speech marker's TF-IDF value (see the color/shade bar on the right). The higher the TF-IDF value, the darker the red color i.e., darker the shade. The lower the TF-IDF value, the lighter the blue color i.e., lighter the shade. The x-axis represents the SHAP value (i.e., the feature importance score). A higher positive value (i.e., higher positive SHAP value) indicates a higher contribution to the prediction of the positive label (i.e., MCI). A lower negative value (i.e., lower negative SHAP value) indicates a higher contribution to the prediction of the negative label (i.e., NC).

According to FIG. 13, the top marker is PAUSE 1202. The corresponding red circles are mainly distributed across the right part of the x-axis with larger positive SHAP values. This means that filler words with higher TF-IDF values significantly contribute to MCI label prediction, suggesting that pauses are more frequently observed among the MCI transcription samples. Similarly, “reaching” 1204, “onto” 1206, and “dish” 1208 with higher TF-IDF values are significant contributors to MCI label prediction, making them the distinguishing markers of MCI compared to NC.

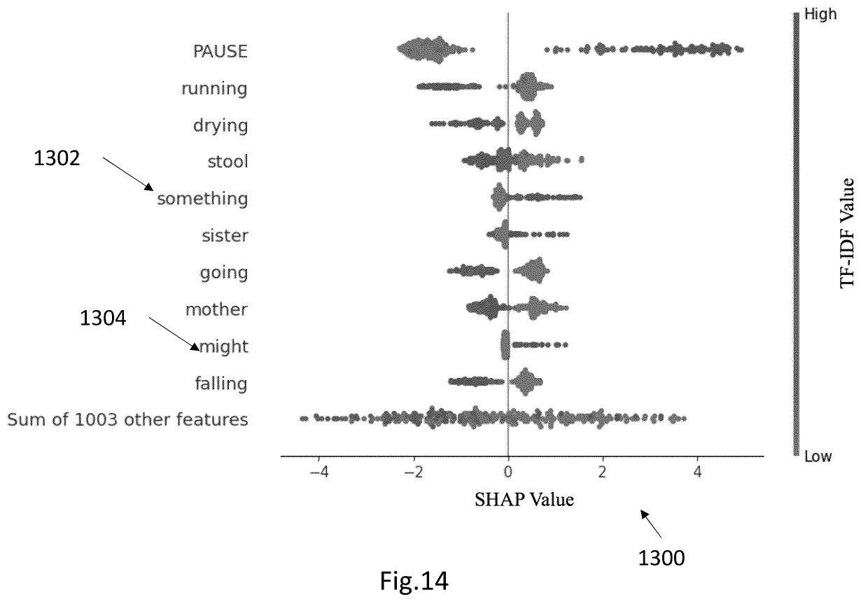

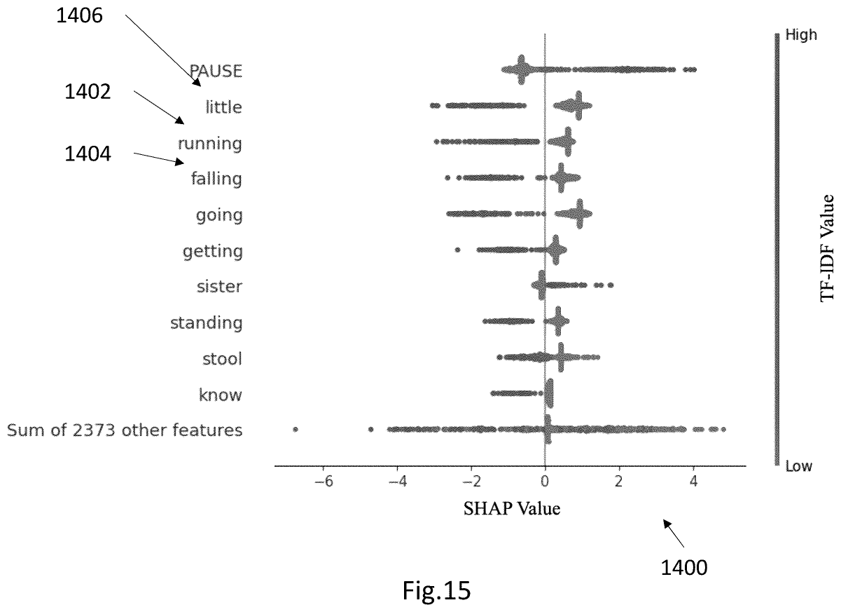

FIGS. 14 and 15 illustrate the top ten speech markers predicting MCI compared to NC based on (1) the best model using the original data and the counterfactual data generation using GPT-4 and (2) the best model using the original data and all data generation using GPT-4. After incorporating the generated data, PAUSE remains the most important marker of MCI prediction.

Referring to plot 1300 of FIG. 14, the top ten speech markers predicting MCI compared to NC based on the best model using the original data and the counterfactual data generation are shown. Each circle in the plot represents one sample. The color or shade of the circle indicates the speech marker's TF-IDF value (see the color/shade bar on the right). The higher the TF-IDF value, the darker the red color. The lower the TF-IDF value, the lighter the blue color. The x-axis represents the SHAP value (i.e., the feature importance score). A higher positive value indicates a higher contribution to the prediction of the positive label (i.e., MCI). A lower negative value indicates a higher contribution to the prediction of the negative label (i.e., NC).

Referring to FIG. 15, the top ten speech markers predicting MCI compared to NC based on the best model using the original data and all data generation are shown on plot 1400. Each circle in the plot represents one sample. The color or shade of the circle indicates the speech marker's TF-IDF value (see the color/shade bar on the right). The higher the TF-IDF value, the darker the red color. The lower the TF-IDF value, the lighter the blue color. The x-axis represents the SHAP value (i.e., the feature importance score). A higher positive value indicates a higher contribution to the prediction of the positive label (i.e., MCI). A lower negative value indicates a higher contribution to the prediction of the negative label (i.e., NC)

Some new markers have emerged after counterfactual data generation, such as “something” 1302 and “might” 1304 (see FIG. 14), suggesting that these speech signals, which were insignificant in the original dataset but may reflect the language deficiencies in MCI subjects, have been amplified in the generated data, with the help of prior knowledge encoded in LLMs when generating MCI-like samples. Furthermore, the top distinctive speech markers separating the MCI samples from NC samples have been highlighted in FIG. 15, suggesting that NC samples are more likely to use verbs, such as “running” 1402, “falling” 1404, and adjectives, such as “little” 1406, compared to MCI samples, which is also evident in previous research where impaired verb fluency is considered a sign of MCI.

The present disclosure provides at least three key advantages. First, many previous speech-based AD detection frameworks have mainly focused on AD dementia. Deviating from these studies, the method and system described have focused on the early detection of dementia among people in the MCI stage, with the aim of reducing MCI prediction biases due to limited and imbalanced data. Second, a novel data generation framework has been developed, leveraging the prior knowledge encoded in LLMs to generate new data samples that can benefit MCI prediction or AD prediction. The described framework exploits novel data generation strategies, including cross-lingual and counterfactual generation, to facilitate out-of-distribution learning and improve prediction performance even when the dataset is limited and imbalanced. Experimental results based on the Pitt corpus have shown that the described LLM-based data generation framework can significantly improve MCI detection sensitivity by up to 38% and F1-score by up to 31%. Third, an explainable AI method, SHAP analysis, has been employed to investigate the key speech markers before and after data generation, providing new insights into speech-based data generation and how it can improve MCI prediction with prior knowledge encoded in LLMs. The explainability analysis provides new insights into speech-based data generation and how it can improve MCI prediction with prior knowledge encoded in LLMs.

The disclosed method and system incorporating new data generation strategies, including cross-lingual and counterfactual generation, are advantageous as they reduce biases in MCI prediction due to the limited and imbalanced data because these two strategies can generate out-of-distribution samples either (1) with the same labels but with more linguistic variations from cross-lingual generation or (2) with opposite labels through counterfactual generation, which requires causal reasoning about the data generation process. These samples are not in the original dataset, making them helpful in model training under low-data settings. This study has particularly attempted to inject causality in LLMs using chain-of-thought prompting. The counterfactual generation has achieved the best improvement compared to other generation strategies (see Table 1000), suggesting certain levels of causal reasoning capability of LLMs, which can be further verified in different scenarios. The system and method are advantageous as they generate datasets that can be used for improved cognitive disease detection or identification.

AI-driven speech-based AD detection studies have shown remarkable performance in detecting AD dementia using audio and text data. However, the detection of AD in its early stages, i.e., MCI onset, remains a challenging task due to the need for sufficient training data and imbalanced diagnosis labels. Recent advancements in GAI and LLMs have provided new insights into building more accurate, unbiased, and reliable AI models in low-data settings with the help of data generation. The disclosed method introduces an LLM-based data generation framework to address the limited and imbalanced data problem. The method (or methods) as described are advantageous because the method comprises proposing two novel data generation strategies to improve MCI prediction. Experimental results (e.g., FIGS. 10 to 15) based on the Pitt corpus from the DementiaBank database have demonstrated that the described framework can significantly enhance MCI detection sensitivity and F1-score by up to 38% and 31%, respectively. Moreover, new speech markers that emerged from data generation have been identified. These findings can help better understand why new data generation can reduce biases in MCI prediction and shed new light on speech-based MCI detection in low-data settings. The disclosed methodology is a general data generation framework that is advantageous as it can be used for improving downstream prediction tasks in other datasets and areas where limited and imbalanced data has presented significant challenges to AI-driven decision-making.

The system and method described herein are advantageous because they significantly improve MCI detection sensitivity, highlighting the potential of LLM-based data generation for improving early-stage AD detection. This disclosure can be used in other disease diagnostic domains where limited and imbalanced speech data are bottlenecks to AI-driven health decision-making. It is an invaluable GAI-driven tool to improve the accuracy and interpretability of speech-based disease diagnostics and beyond.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present disclosure are either wholly implemented by computing system or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

Any reference to prior art contained herein is not to be taken as an admission that the information is common general knowledge, unless otherwise indicated.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

1. A computer implemented for generating data for use in cognitive disease detection comprising the steps of:
receiving initial disease related data,
generating improved disease related data by applying a generative AI data generation framework to the initial disease related data, and;
storing the improved disease related data samples, and;
wherein the method is configured to generate the improved data for use in mild cognitive impairment (MCI) detection in patients.

2. The computer implemented method of claim 1 wherein the step of generating improved data samples comprises generating improved disease related data by applying one or more large language models to the initial disease related data.

3. The computer implemented method of claim 2 wherein step of generating the improved disease related data comprises applying one or more of the following data generation strategies:
a) observational data generation
b) cross lingual data generation
c) counterfactual data generation.

4. The computer implemented method of claim 3 wherein the step of generating the improved disease related data comprises applying each of the following data generation strategies a) observational data generation, b) cross lingual data generation and c) counterfactual data generation.

5. The computer implemented method of claim 4 wherein the improved disease related data comprises out-of-distribution samples comprising the same labels but with more linguistic variations from cross lingual data generation and/or out-of-distribution samples with opposite labels through counterfactual generation.

6. The computer implemented method of claim 3 wherein each data generation strategy is implemented by a separate generative AI model executed on a computing apparatus.

7. The computer implemented method of claim 6 wherein each data generation strategy is implemented by inputting a prompt to a generative AI model corresponding to a data generation strategy, wherein each prompt comprises a system message and a user message.

8. The computer implemented method of claim 7 wherein each generative AI model comprises a large language model.

9. The computer implemented method of claim 7 wherein the system message comprises a two-part message, a first part of the system message provides background information for data generation, and a second part of the system message provides detailed instructions for new data generation.

10. The computer implemented method of claim 9 wherein the user message comprises input information for data generation, wherein the input information comprises one or more of: transcription text, diagnosis labels, age, gender, race, education and MMSE information.

11. A system generating data for use in cognitive disease detection comprising:
a computing apparatus,
the computing apparatus comprising a processing unit, a memory unit and a user interface, the processing unit is operatively coupled to the memory unit and the user interface,
the computing apparatus is configured to:
receiving initial disease related data,
generating improved disease related data by applying a generative AI data generation framework to the initial disease related data,
storing the improved disease related data samples, and;
wherein the step of generating the improved disease related data comprises applying each of the following data generation strategies a) observational data generation, b) cross lingual data generation and c) counterfactual data generation.

12. The system of claim 11 wherein the system is configured to generate the improved data for use in mild cognitive impairment (MCI) detection in patients.

13. The system of claim 11 wherein the computing apparatus is configured to:
generate improved disease related data by applying one or more large language models to the initial disease related data, and;
apply each of the following data generation strategies a) observational data generation, b) cross lingual data generation and c) counterfactual data generation.

14. The system of claim 13 wherein the improved disease related data comprises out-of-distribution samples comprising the same labels but with more linguistic variations from cross lingual data generation and/or out-of-distribution samples with opposite labels through counterfactual generation.

15. The system of claim 14 wherein the computing apparatus is configured to execute a separate generative AI model to implement each data generation strategy,
wherein each data generation strategy is implemented by inputting a prompt to a generative AI model corresponding to a data generation strategy, wherein each prompt comprises a system message and a user message, and
each generative AI model comprising a large language model (LLM).

16. The system of claim 15 wherein the system message comprises a two-part message, a first part of the system message provides background information for data generation, and a second part of the system message provides detailed instructions for new data generation, and; wherein the user message comprises input information for data generation, wherein the input information comprises one or more of: transcription text, diagnosis labels, age, gender, race, education and MMSE information.

17. A computer implemented method for generating data for use in mild cognitive impairment (MCI) detection comprising the steps of:
receiving, by a processor, initial disease-related data comprising at least a first transcription text of a subject's speech and an associated first diagnosis label,
generating, by the processor using a generative AI framework comprising one or more large language models (LLMs), improved disease related data by applying a plurality of data generation strategies to the initial disease-related data, the plurality of data generation strategies comprising:
(a) observational data generation,
(b) cross-lingual data generation, and;
(c) counterfactual data generation, and;
wherein the cross lingual data generation and the counterfactual data generation are applied to generate out-of-distribution samples,
storing, in a memory, the improved disease-related data.

18. The computer implemented method of claim 17, wherein the improved disease related data comprises out-of-distribution samples comprising the same labels but with more linguistic variations from cross lingual data generation and/or out-of-distribution samples with opposite labels through counterfactual generation.

19. The computer implemented method of claim 17 wherein each data generation strategy is implemented by inputting a prompt to a generative AI model corresponding to a data generation strategy, wherein each prompt comprises a system message and a user message,
wherein each generative AI model comprises a large language model,
wherein the system message comprises a two-part message, a first part of the system message provides background information for data generation, and a second part of the system message provides detailed instructions for new data generation, and;
wherein the user message comprises input information for data generation, wherein the input information comprises one or more of: transcription text, diagnosis labels, age, gender, race, education and MMSE information.

20. The computer implemented method of claim 17 comprising the steps of:
training a text-based disease classification model using a training dataset that includes the initial disease-related data and the improved disease-related data,
wherein training the text-based disease classification model comprises: vectorizing transcription texts in the training dataset to generate term frequency-inverse document frequency (TF-IDF) vectors; and constructing an extreme Gradient Boosting (XGBoost) model using the TF-IDF vectors as inputs,
performing a feature importance analysis on the trained text-based disease classification model to identify speech markers predictive of MCI, wherein the feature importance analysis is performed using Shapley Additive explanations (SHAP),
wherein the observational data generation comprises instructing the LLM to:
first, output an explanation of linguistic characteristics of the first transcription text corresponding to the first diagnosis label; and second, output the second transcription text based on the explanation and;
wherein the counterfactual data generation comprises instructing the LLM to generate the third, counterfactual transcription text while maintaining demographic information associated with the subject unchanged from the initial disease-related data.
